(19) United States
(12) Patent Application Publication
(10) Pub. No.: US 2022/0185802 A1
(43) Pub. Date: Jun. 16, 2022

(54) BENZOXAZINE MONOMERS AND POLYMERIC COATINGS

(71) Applicants: LUXEMBOURG INSTITUTE OF SCIENCE AND TECHNOLOGY (LIST), Esch-sur-Alzette (LU); UNIVERSITY OF MANCHESTER, Manchester (GB)

(72) Inventors: Pierre VERGE, Esch-sur-Alzette (LU); Nicola TIRELLI, Manchester (GB); Julio Manuel RIOS DE LA ROSA, Madrid (ES); Philippe DUBOIS, Ciplet (BE)

(21) Appl. No.: 17/441,844

(22) PCT Filed: Mar. 17, 2020
(86) PCT No.: PCT/EP2020/057335

§ 371 Date: Sep. 22, 2021

(30) Foreign Application Priority Data
Mar. 22, 2019 (LU) LU101161

(51) Int. Cl. C07D 413/06; C08G 73/02; C09D 179/04; A61L 27/34; A61L 27/54; A61L 29/08; A61L 29/16

(52) U.S. Cl. CPC C07D 413/06; C08G 73/0233; C09D 179/04; A61L 2300/41; A61L 27/54; A61L 29/085; A61L 29/16; A61L 27/34

(57) ABSTRACT

Monomer(s) based on a 3,4-dihydro-2H-1,3-benzoxazine derivative. Alternatively, a composition comprising a mixture of the monomer(s) and of a second benzoxazine derivative selected from the group consisting of monofunctional amines bridged with diphenolic compounds, monophenolic compounds bridged with diamines and diamines bridged by diphenolic compounds, or a mixture thereof. These compounds are suited for the preparation of benzoxazine derivative heterogeneous polymers, for example having the shape of coatings or films, exhibiting enhanced anti-inflammatory and/or anti-oxidant properties. A material with a coated substrate with the heterogeneous polymer may be a part of implantable materials, such as implantable metallic apparatus such as a catheter, a metallic implant, or a metallic prosthesis, biologically compatible. These implantable materials may be safely used because they are preventing any inflammatory and/or oxidative reaction(s) from a host into which the coated substrate is grafted or implanted.

The present invention is the US national stage under 35 U.S.C. § 371 of International Application No. PCT/EP2020/057335 which was filed on Mar. 17, 2020, and which claims the priority of application LU 101161 filed on Mar. 22, 2019, the content of which (text, drawings and claims) are incorporated here by reference in its entirety.

### FIELD

The invention is directed to benzoxazine derivative compounds useful for the preparation of anti-inflammatory benzoxazine derivative polymeric coatings.

### BACKGROUND

Synthetic polymer coatings are used extensively in modern medical devices and implants because of their material versatility and processability. However, implantation of these materials into the body elicits a strong inflammatory host response that significantly limits the integration and biological performance of devices (J. Diab. Sci. Tech., 2008, 2(6), 984-994). Various approaches used for modifying material surfaces to modulate inflammatory responses such as non-fouling surface treatments (passive strategy) and delivery of anti-inflammatory agents (active strategy) exist.

Prior art patent document published WO 2005/039443 A2 relates to the implantation of a stent at an injured region in combination with the delivery of a tocopherol or tocotrienol. Among other advantages, these molecules exhibit anti-inflammatory properties.

The delivery of anti-inflammatory agents has the drawback that the desired properties can only be given for a limited period of time. Moreover, the use of tocopherol or tocotrienol derivative is not readily straightforward since those compounds as such polymerize hardly and deposit thereof onto substrates has to be optimised.

### SUMMARY

The invention has for technical problem to provide new benzoxazine derivative compounds, advantageously used as precursors for the preparation of anti-inflammatory and/or antioxidant benzoxazine polymeric coatings, in a method for rendering the surface of an implantable object containing the coating less susceptible on the long term to cause an inflammatory and/or antioxidant reaction from the host in which the object is implanted or grafted.

The invention is directed to an anti-inflammatory and/or antioxidant monomer based on a 3,4-dihydro-2H-1,3-benzoxazine derivative having the formula (1)

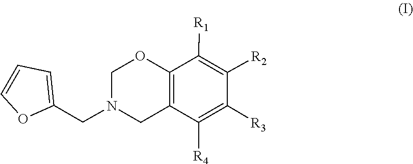

either wherein
R₄=CH₃
R₂ and R₃ together represent

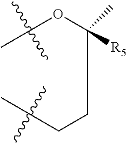

with R₅=

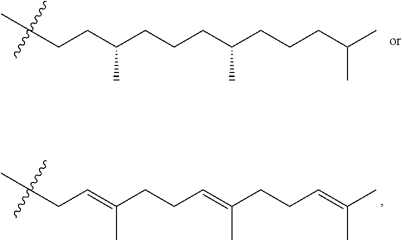 [SMILES: *CC[C@@H](C)CCC[C@@H](C)CCCC(C)C ; *C/C=C(\C)CC/C=C(\C)CCC=C(C)C]

and
R₁=H, or CH₃,
or wherein
R₄ and R₃ together represent

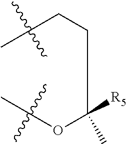

with R₅=

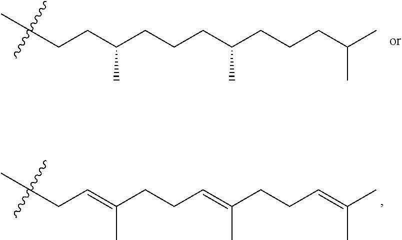 [SMILES: *CC[C@@H](C)CCC[C@@H](C)CCCC(C)C ; *C/C=C(\C)CC/C=C(\C)CCC=C(C)C]

and
R₂=R₁=CH₃,
or a mixture thereof.

The Applicant has surprisingly shown that these monomers, exhibiting anti-inflammatory and/or anti-oxidant effects, but not limited to, are very useful for the preparation of benzoxazine derivative heterogeneous polymers, for example having the shape of coatings or films, exhibiting enhanced anti-inflammatory and/or anti-oxidant properties.

Anti-inflammatory and/or anti-oxidant properties of both monomers, as starting materials, and heterogeneous polymers obtainable by a polymerisation process described below, are maintained. This is one the main purpose of the invention. Besides, coatings with the heterogeneous polymers are also compatible with cells of the body, i.e. devoid of toxicity. This allows the development of implantable materials, such as implantable metallic apparatus such as a catheter, a metallic implant, or a metallic prosthesis which may not cause inflammatory effects or the effects being essentially avoided when used in a human or animal body. Besides, coatings with the heterogeneous polymers may advantageously exhibit a radical scavenging activity, and ensuing anti-oxidant properties. The use of the benzoxazine chemistry also allows the self-condensation of the monomer(s), avoiding the use of a catalyst or a hardener (as for epoxy resins), which generally wreak havoc on the human health.

Moreover, the derivation of the tocopherol, tocotrienol, cardanol and/or eugenol derivatives, not limited to, into polymers capable to be deposited onto a surface of a substrate is investigated and is another aspect of the invention. Among the various isomers of benzoxazines, 3,4-dihydro-2H-1,3-benzoxazine derivatives monomer of the invention are of interest for the development of polymeric materials owing to their cross-linking via polymerisation, especially ring-opening polymerization. In other words, the polymerization is leading to a three-dimensional cross-linked polymer. It is known that benzoxazines are synthesized by heating a mixture of the appropriate amine, phenol and formaldehyde. Especially, the Applicant has implemented a solventless synthesis procedure in order to prevent the formation of oligomers (see U.S. Pat. No. 5,543,516 patent) which is one of the advantages of the current invention.

Another aspect of the invention is a composition comprising a mixture of a first monomer consisting of the anti-inflammatory and/or antioxidant 3,4-dihydro-2H-1,3-benzoxazine derivative monomer of the invention, or a mixture thereof, and a second benzoxazine derivative selected from the group consisting of monofunctional amines bridged with diphenolic compounds, monophenolic compounds bridged with diamines and diamines bridged by diphenolic compounds, or a mixture thereof.

The second benzoxazine derivative is very useful for implementing a self-polymerisation of the first monomer. This could be explained by the fact that the first monomer bears one oxazine ring (mono-functional benzoxazine) leading by self-polymerisation to a heterogeneous polymer that is less preferred to realise a coating. Thus, owing to the second benzoxazine derivative, bearing at least two oxazine rings, the composition is especially suited for the preparation of a material coated by the heterogeneous polymer with improved properties, such as adherence on various substrates like steel or glass based material or the like.

Besides, it is not necessary for the second benzoxazine derivative to exhibit any significant anti-oxydant and/or anti-inflammatory effects. It is used advantageously used to enable or even enhance the polymerisation, and it is specifically selected not to impair the anti-inflammatory and/or anti-oxidant activities of the tocopherol, tocotrienol, cardanol and/or eugenol derivatives or of the first benzoxazine derivative monomer.

In various instances, in the composition, the second benzoxazine derivative has a concentration equal or superior to 50% in weight in comparison to the concentration of the first monomer. Such a concentration of the second benzoxazine derivative does improve the coatings properties on various substrates in terms of, for example, adherence, hardness and abrasion resistance. In various instances, the concentration of the second benzoxazine derivative is within the range of 55% in weight to 90% in weight, for example the concentration is within the range of 60% in weight to 80%. These last two concentrations ranges allow even better performances relative to hardness, adherence and/or abrasion resistance.

In various instances, the second benzoxazine derivative is a compound of formula A

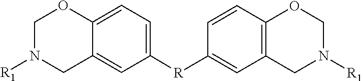

wherein R is a —CH₂—, —C(CH₃)₂—, SO₂, —C(CF₃)₂—, —C(CH₃)(C₆H₅)—, —C(CH₃)(C₂H₅)—, —C(C₆H₅)₂—, —CHCH₃—, —C₆H₁₀— or —C(CH₃)CH₂CH₂COOH— group;
R₁ is a —CH₂CH₂OH, vinyl, methyl, ethyl, propyl, isopropyl, butyl, hexyl, cyclohexyl, fluorene, phenylacetylene, phenyl propargyl ether, benzonitrile, furfuryl, phenylene or —(CH₂)₁₇CH₃ group;

According to another exemplary embodiment, the second benzoxazine derivative is a compound of formula B

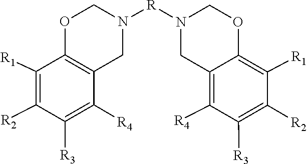

either wherein
R is a —(CH)₂—, —(CH)₄—, —(CH)₆—, —(CH)₆—, —(CH)₁₀—, —(CH)₁₂—, —(CH)₁₄—, —(CH)₁₆— group or

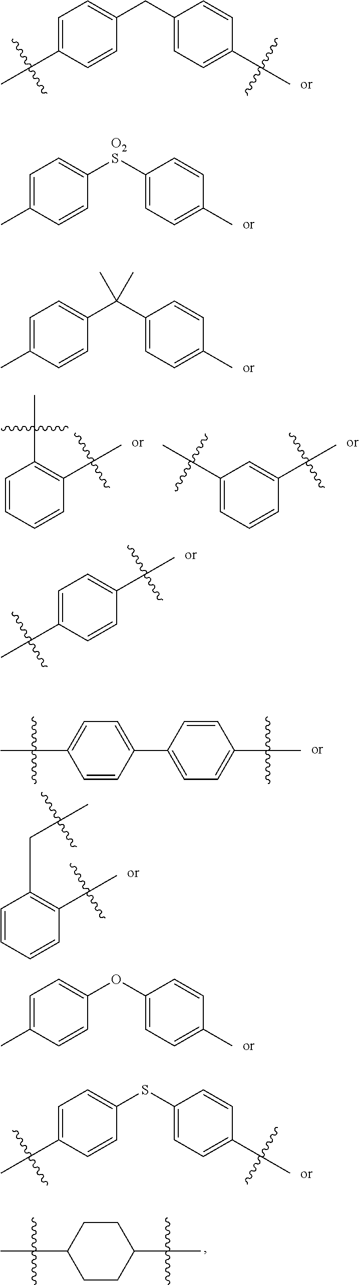

and wherein R₁, R₂, R₃, R₄ are selected from the group consisting of
R₁=R₂=R₃=R₄=H,
R₁=OCH₃, R₂=R₄=H, R₃=CH₂CH═CH₂,
R₁=OCH₃, R₂=R₄=H, R₃=CH═CHCH₃,
R₁=OCH₃, R₂=R₄=H, R₃=CHO,
R₁=OCH₃, R₂=R₃=R₄=H,
R₁=OCH₃, R₂=R₄=H, R₃=CHO,
R₁=OCH₃, R₂=R₄=H, R₃=CH₂CH₂COOH,
R₁=R₂=R₄=H, R₃=CH₂CH₂COOH,
R₁=R₂=R₄=H, R₃=CH═CHCOOH, and
R₁=OCH₃, R₂=R₄=H, R₃=CH═CHCOOH,
or wherein
R₃=R₄=H,

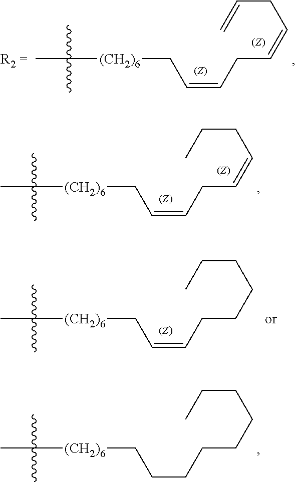

### and R₁=COOH,

or wherein
R₁=H, R₂=OH, R₃=H
and

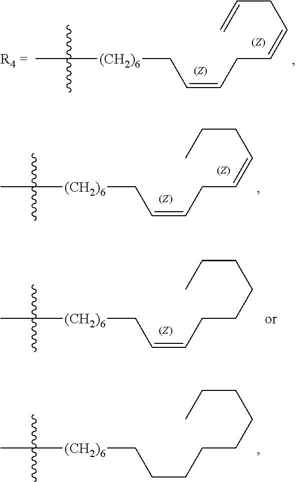

or wherein

### R₁=H,

and

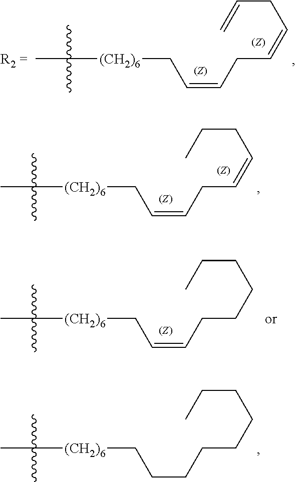

and R₃=H, R₄=OH,
or wherein
R₁=CH₃, R₂=OH, R₃=H

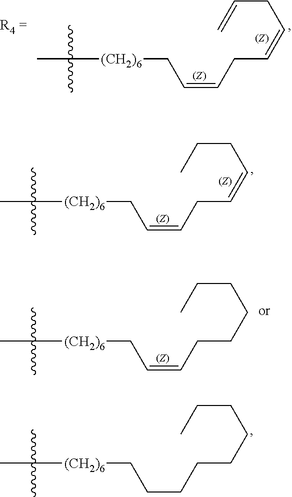

or wherein
R₁=R₃=R₄=H, and

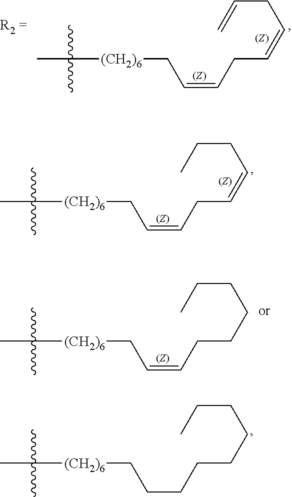

or wherein
R₁=R₃=H
R₂=—CH₂HC═CH₂, and R₄=OCH₃;
or
R₁=R₃=H
R₂=—CH═HCCH₃ and
R₄=OCH₃;
or R₁=R₂=R₄=H and

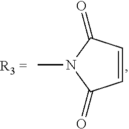

or R₁=R₂=R₃=H and R₄=CH₂CH═CH₂
or R₁=R₂=R₄=H and R₃=CH₂CH═CH₂
or R₁=R₃=R₄=H and R₂=CH₂CH═CH₂.

According to an alternate embodiment, the second benzoxazine derivative is a compound of formula C

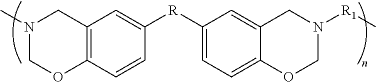

wherein R is a —CH₂—, —C(CH₃)₂—, SO₂, —C(CF₃)₂—, —C(CH₃)(C₆H₅)—, —C(CH₃)(C₂H₅)—, —C(C₆H₅)₂—, —CHCH₃—, —C₆H₁₀— or —C(CH₃)CH₂CH₂COOH— group;
   and either within
   n is an integer from 1 to 10;
   R₁ is a —(CH)₂—, —(CH)₄—, —(CH)₆—, —(CH)₈—, —(CH)₁₀—, —(CH)₁₂—, —(CH)₁₄—, or —(CH)₁₈— group; or

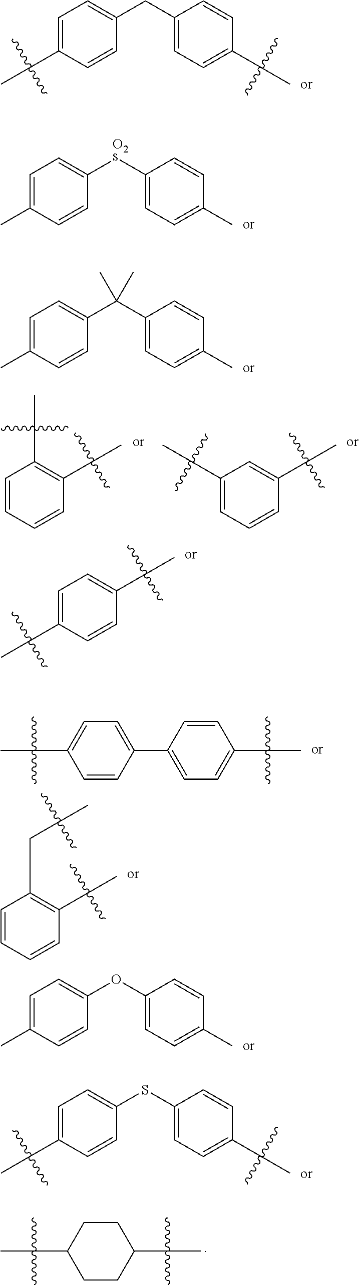

In various instances, the second benzoxazine derivative is a compound of formula

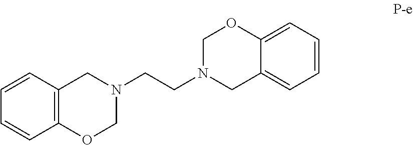 [SMILES: c1ccc2c(c1)CN(CCN1COc3ccccc3C1)CO2]

According to the invention, the second benzoxazine derivative is either a monomer derivative or a polymer derivative.

Another aspect of the invention concerns a process for producing an anti-inflammatory and/or antioxidant material comprising a substrate coated with an anti-inflammatory and/or antioxidant heterogeneous polymer based on 3,4-dihydro-2H-1,3-benzoxazine, the process comprising the steps of:
(a) depositing an anti-inflammatory and/or antioxidant monomer selected from the group consisting of at least any one monomer of above formulas and of further formula:

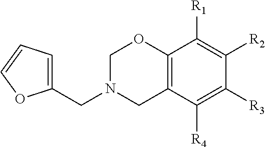

either wherein
   R₃=R₄=H,

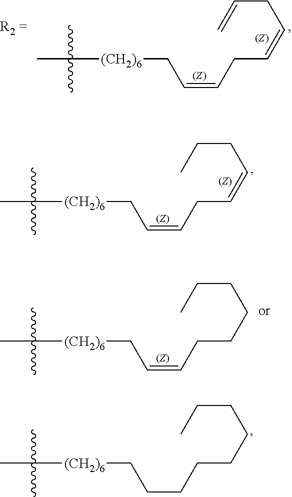

### and R₁=COOH,

or wherein
R₁=H, R₂=OH, R₃=H
and

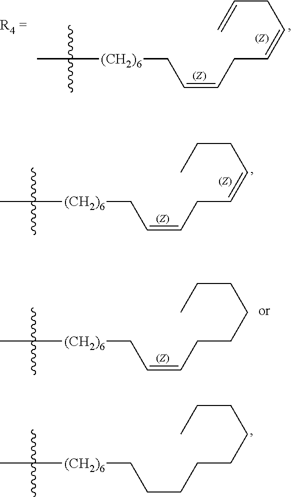

or wherein

### R₁=H,

and

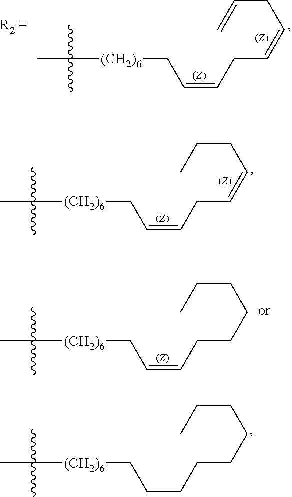

and R₃=H, R₄=OH,
or wherein
R₁=CH₃, R₂=OH, R₃=H

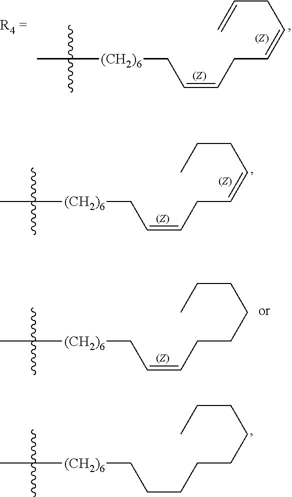

or wherein
R₁=R₃=R₄=H, and

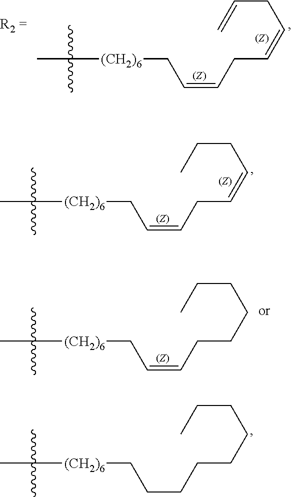

or wherein
R₂=R₄=H
R₃=—CH₂HC═CH₂ and
R₁=OCH₃;
or
R₂=R₄=H
R₃=—CH═HCCH₃ and
R₁=OCH₃,
or a mixture thereof, onto the substrate,
(b) heating a deposit obtained through step (a) at a predetermined temperature and for a predetermined duration to polymerize the monomer to obtain the material.

The Applicant has shown that this process, wherein the polymerization is carried out especially via a ring-opening polymerisation, is advantageously effective, in one hand, to prepare the heterogeneous polymeric compounds (three-dimensional cross-linked polymer) for their not limitative use for example as coatings on various substrate(s), and, in the other hand, for maintaining or not to impair the anti-inflammatory and/or anti-oxidant properties of the starting material (monomer based on a 3,4-dihydro-2H-1,3-benzoxazine derivative) during the polymerisation and of the heterogeneous polymer based on a 3,4-dihydro-2H-1,3-benzoxazine derivative. These both aspects are purposes of the invention. In the context of the invention, “heterogeneous polymer based on a 3,4-dihydro-2H-1,3-benzoxazine derivative” should be understood as resulting from the monomer bearing 1,3 benzoxazine moiety, but the polymer is no more bearing such benzoxazine moieties.

In the context of the invention too, the expressions “a heterogeneous polymer based on 3,4-dihydro-2H-1,3-benzoxazine derivative”, “heterogeneous polymer”, “benzoxazine derivative heterogeneous polymer” and “heterogeneous polymer having anti-inflammatory and/or anti-oxidant properties” have the same meanings.

According to the process, the heating to a predetermined temperature for a predetermined duration is advantageously carried out for transforming at least 80% of the starting monomer, in various instances at least 90%, for example 99.9% of the starting monomer into the anti-inflammatory and/or antioxidant heterogeneous polymer.

The implementation of the process allows the manufacture of coatings or films including the heterogeneous polymeric compounds exhibiting enhanced anti-inflammatory and/or anti-oxidant properties. In various instances, the coating or films may be obtained through direct implementation of the process, avoiding any need of further reshaping of the heterogeneous polymer on the surface of the substrate. These coatings may also very especially be compatible with cells of the body, i.e. devoid of toxicity. This allows the development of implantable materials, such as implantable metallic apparatus, biologically compatible, such as a catheter, a metallic implant, or a metallic prosthesis which may then not cause inflammatory effects or the effects being essentially avoided when used in a human or animal body. The use of the benzoxazine chemistry also allows the self-condensation of the monomer(s), avoiding the use of a catalyst or a hardener (as for epoxy resins), which is detrimental to the human health.

According to an exemplary embodiment, step (b) of heating may be performed at a temperature within a range of from 100° C. to 250° C. In various instances, the heating temperature is within a range of from 120° C. to 200° C., particularly of from 150° C. to 180° C.

The duration of the polymerisation may mainly depend upon the polymerisation temperature, the polymerisation duration may advantageously be of from 30 min to 4 h.

The depositing step (step (a)) may cover at least a part of the substrate or fully cover it. The deposition step (a) is in various instances carried out so as to form a layer presenting a thickness of from 100 μm to 2 mm, for example from 100 μm to 1 mm. Best anti-inflammatory and/or anti-oxidant effects are obtained when thickness of the coating or the film is from 100 μm to 500 μm.

According to a very advantageous embodiment, the anti-inflammatory and/or antioxidant monomer based on a 3,4-dihydro-2H-1,3-benzoxazine derivative being a first monomer, the process comprises a step of depositing a second benzoxazine derivative selected from the group consisting of monofunctional amines bridged with diphenolic compounds, monophenolic compounds bridged with diamines and diamines bridged by diphenolic compounds, or a mixture thereof, onto the substrate deposited with the first monomer during step (a) for generating a mixture, before the step (b) of heating.

The resulting mixture is in various instances a bi-layer system on the substrate, i.e wherein a first layer of the first monomer is deposited onto the substrate, followed by a deposition step of the second benzoxazine derivative. This results on a mixture which is at least partly homogeneous at least at the layers interface.

In the context of the invention, the first monomer is pure, or it may be diluted just enough by an appropriate solvent for obtaining a composition of the first monomer.

The second benzoxazine derivative is very useful for enhancing or even enhance a self-polymerisation (step (b)) of the first monomer. This could be explained by the fact that the first monomer bears one oxazine ring (mono-functional benzoxazine) leading by self-polymerisation, i.e. ring-opening polymerization, to a three-dimensional cross-linked polymer, the heterogeneous polymer of the invention, that is optional to realise a coating. Thus, owing to the second benzoxazine derivative bearing at least two oxazine rings, the process according to the invention allows the preparation of a coating, based on the heterogeneous polymer, with improved properties, such as adherence on various substrates such as steel or glass based material or the like. Advantageously, the second benzoxazine derivative is a compound of formula A or B, as defined above, or a mixture thereof.

In various instances, the process includes a step of adding a second benzoxazine derivative, selected from the group consisting of monofunctional amines bridged with diphenolic compounds, monophenolic compounds bridged with diamines and diamines bridged by diphenolic compounds, or a mixture thereof, to the anti-inflammatory and/or antioxidant monomer based on a 3,4-dihydro-2H-1,3-benzoxazine derivative being a first monomer, and a step of mixing to form a resulting composition of the first monomer and second benzoxazine derivative, the resulting composition being deposited onto the substrate in the step (a).

In various instances, the second benzoxazine derivative, for example in the above resulting composition, has a concentration equal or superior to 50% in weight in comparison to the concentration of the first monomer. Such a concentration of the second benzoxazine derivative does improve the coatings properties on various substrates in terms of, for example, adherence. For example, the concentration of second benzoxazine derivative is within the range of 55% in weight to 90% in weight, in various instances the concentration is within the range of 60% in weight to 80%. These concentrations ranges are effective to improve, adherence, hardness and abrasion resistance of the coating onto various substrates.

According to an exemplary embodiment, the step (a) of depositing may be performed by melting or by dissolving the first monomer or the second benzoxazine derivative or the resulting composition of the first monomer and second benzoxazine derivative, thereby a homogeneous mixture is obtained. The melting process of monomer(s), known in the art, is however advantageous, preventing the use of possibly toxic organic solvents. Moreover, the absence of solvents give better coatings characteristics, such as recovering ability onto substrates, homogeneity, reduced or no mini-fractures observed on coatings. When the melting process is used, typical melting temperatures are of from 100° C. to 120° C., for example of from 100° C. to 110° C.

Alternately, the step (a) of depositing may be performed in an aprotic solvent, leading to the homogeneous mixture, and are not limited, with the proviso that the process could be performed. For example, aromatic hydrocarbons, for example toluene, and halogenated hydrocarbons, for example, chloroform, carbon tetrachloride, and dichloromethane may be used. Ketones solvents could also be employed, like acetone.

The substrate that is used for performing the process may be for example steel, glass or polymeric based material or the like, with the proviso that the heating step (b) is not adversely affecting the substrate.

Advantageously, the step (b) of heating is performed in presence of a Lewis acid type catalyst, the catalyst being in various instances selected from the group consisting of PCl₅, PCl₃, POCl₃, TiCl₄, AlCl₃ and CH₃OTf, or a mixture thereof. The use of such Lewis acid catalyst allows the implementation of step (c) at temperatures lower than 180° C., for example at temperatures of from 150° C. to 180° C. Lowering the temperature of the step (c) is very desired to avoid any thermal degradation of monomers and/or the substrate.

The process may further comprise a step (c) of sterilizing the heterogeneous polymer on the substrate, the step (c) being in various instances performed by soaking the heterogeneous polymer on the substrate into a solution of a primary alcohol, such as methanol or ethanol. The solution may be consisting of pure primary alcohol or a diluted solution thereof in water, typically containing at least 60% in volume of the alcohol.

In various instances, the process may also include, prior to step (a), a step of adding an adhesion promoter, as a coating or a film, on the substrate. Examples of suitable adhesion promoters include amino-silane coupling agents such as (3-aminopropyl)-triethoxysilane, (3-aminopropyl)-diethoxy-methylsilane, (3-aminopropyl)-dimethyl-ethoxysilane or (3-aminopropyl)-trimethoxysilane, or a mixture thereof.

The invention further concerns an anti-inflammatory and/or antioxidant material comprising a substrate with an anti-inflammatory and/or antioxidant heterogeneous polymer based on 3,4-dihydro-2H-1,3-benzoxazine coating or film, obtainable by the process of the invention.

In the material, the heterogeneous polymer based on 3,4-dihydro-2H-1,3-benzoxazine derivative obtainable by the process of the invention, in various instances presents a mechanical relaxation temperature corresponding to the maximum of the loss factor of from 100° C. to 300° C., for example of from 100° C. to 250° C., especially of from 100° C. to 150° C.

The coating or the film is presenting a thickness of from 100 μm to 2 mm, for example of from 100 μm to 1 mm. Best anti-inflammatory and/or anti-oxidant effects are obtained when thickness of the coating or the film is from 100 μm to 500 μm.

The hardness of a coating or a film is measured using the Pencil Hardness Tester Elcometer 501, in accordance with the following standard ASTM D 3363. A scratch on the coating was typically not observed using pencil with a hardness lower than 5H. The coating also in various instances exhibits a mechanical relaxation temperature corresponding to the maximum of the loss factor of from 100° C. and 300° C., for example of from 100° C. to 250° C., especially of from 100° C. to 150° C.

Still another aspect of the invention is concerning the use of the monomer based on a 3,4-dihydro-2H-1,3-benzoxazine derivative of any one of formulas defined above, or mixture thereof, or the above composition, as a precursor for obtaining a heterogeneous polymer having anti-inflammatory and/or anti-oxidant properties.

The invention also relates to a coated substrate of a material, as above defined, comprising a film or a coating of a heterogeneous polymer prepared from the monomer based on a 3,4-dihydro-2H-1,3-benzoxazine derivative of any one of formulas defined above or a heterogeneous polymer prepared from the composition. The raw substrate, before coating, may be a steel or glass based material or the like.

The coated substrate of the material may also in various instances include an adhesion promoter, as a coating or a film, between the substrate and the film or a coating prepared from the monomer based on a 3,4-dihydro-2H-1,3-benzoxazine derivative. Examples of suitable adhesion promoters include amino-silane coupling agents such as (3-aminopropyl)-triethoxysilane, (3-aminopropyl)-diethoxy-methylsilane, (3-aminopropyl)-dimethyl-ethoxysilane or (3-aminopropyl)-trimethoxysilane, or a mixture thereof.

The material is advantageously a part of implantable materials, such as implantable metallic apparatus such as a catheter, a metallic implant, or a metallic prosthesis, biologically compatible. These implantable materials may be safely used because they are preventing any inflammatory and/or oxidative reaction(s) from a host into which the coated substrate is grafted or implanted.

The invention also relates to an anti-inflammatory and/or antioxidant material according of the invention for use for the treatment or the prevention of inflammatory and/or oxidant disease(s).

The invention also relates to a use of the anti-inflammatory and/or antioxidant monomer based on 3,4-dihydro-2H-1,3-benzoxazine derivative as a precursor for obtaining an anti-inflammatory and/or anti-oxidant heterogeneous polymer of the invention.

### DRAWINGS

FIG. 1 is an exemplary illustration of a chemical structure of the monomers 1-13 (comparative example) used in various embodiments of the present invention.

FIG. 2 is an exemplary illustration of a chemical structure of monomers 7b, 8b, 9b, 10b, 11b, 12b and 13b (comparative example), in accordance with various embodiments of the present invention.

FIG. 3 is an exemplary illustration of a chemical structure of monomers 7c, 8c, 9c, 10c, 11c, 13c and (comparative example), in accordance with various embodiments of the present invention.

FIG. 4 is an exemplary illustration of a chemical structure of monomers 7d, 8d, 9d, 10d, 11d, and 13d (comparative example), in accordance with various embodiments of the present invention.

FIG. 5 is an exemplary illustration of a chemical structure of P-e (second benzoxazine derivative), in accordance with various embodiments of the present invention.

FIG. 6 is an exemplary illustration of a ¹H NMR (600 MHz, CDCl₃) spectrum of the monomer 1, in accordance with various embodiments of the present invention.

FIG. 7 is an exemplary illustration of a ¹³C NMR (150 MHz, CDCl₃) spectrum of the monomer 1, in accordance with various embodiments of the present invention.

FIG. 8 is an exemplary illustration of a mass spectrum (MALDI-Tof/MS) of the monomer 1, in accordance with various embodiments of the present invention.

FIG. 9 is an exemplary illustration of an IR spectrum of the monomer 1, in accordance with various embodiments of the present invention.

FIG. 10 is an exemplary illustration of a DSC curve of the monomer 1, in accordance with various embodiments of the present invention.

FIG. 11 is an exemplary illustration of a DSC curves of the composition comprising the monomer 1 and P-e, in accordance with various embodiments of the present invention.

FIG. 12 is an exemplary illustration of a coating of P-e on a steel coverslip that has been treated with NaOH, in accordance with various embodiments of the present invention.

FIG. 13 is an exemplary illustration of a coating of the composition (monomer 1+P-e) on a steel coverslip that has been treated with NaOH, in accordance with various embodiments of the present invention.

FIG. 14 is an exemplary illustration of a coating of the composition (monomer 1+P-e) on a steel coverslip that has not been treated with NaOH, in accordance with various embodiments of the present invention.

FIG. 15 is an exemplary illustration of a rheogram featuring the polymerization of the composition (monomer 1+P-e), in accordance with various embodiments of the present invention.

FIG. 16 is an exemplary illustration of a rheogram featuring the mechanical relaxation temperature of the cross-linked composition (monomer 1+P-e), in accordance with various embodiments of the present invention.

FIG. 17 is an exemplary illustration of an effect of the substrate on the metabolic activity in RAW 264.7 (left) and THP-1 (right) macrophages, in accordance with various embodiments of the present invention.

FIG. 18 is an exemplary illustration of an effect of the substrate on the TNF-α production in RAW 264.7 (left) and THP-1 (right) macrophages, in accordance with various embodiments of the present invention.

FIG. 19 is an exemplary illustration of a measurement of the inflammation inhibitory activity of 5 samples, in accordance with various embodiments of the present invention.

FIG. 20 is an exemplary illustration of a measurement of the pro-inflammatory activity of 5 samples, in accordance with various embodiments of the present invention.

FIG. 21 is an exemplary illustration of a measurement of the toxicity of 5 samples, in accordance with various embodiments of the present invention.

### DETAILED DESCRIPTION

### Preparation of the Monomers

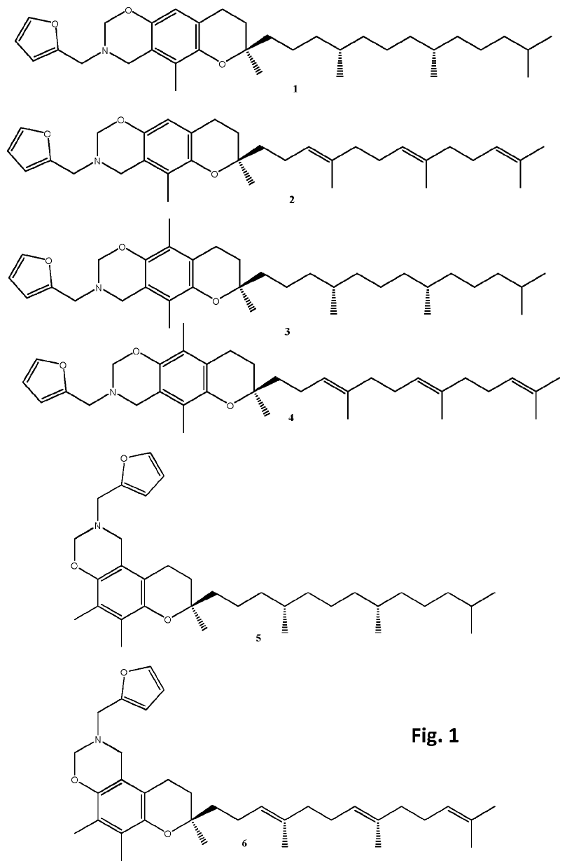

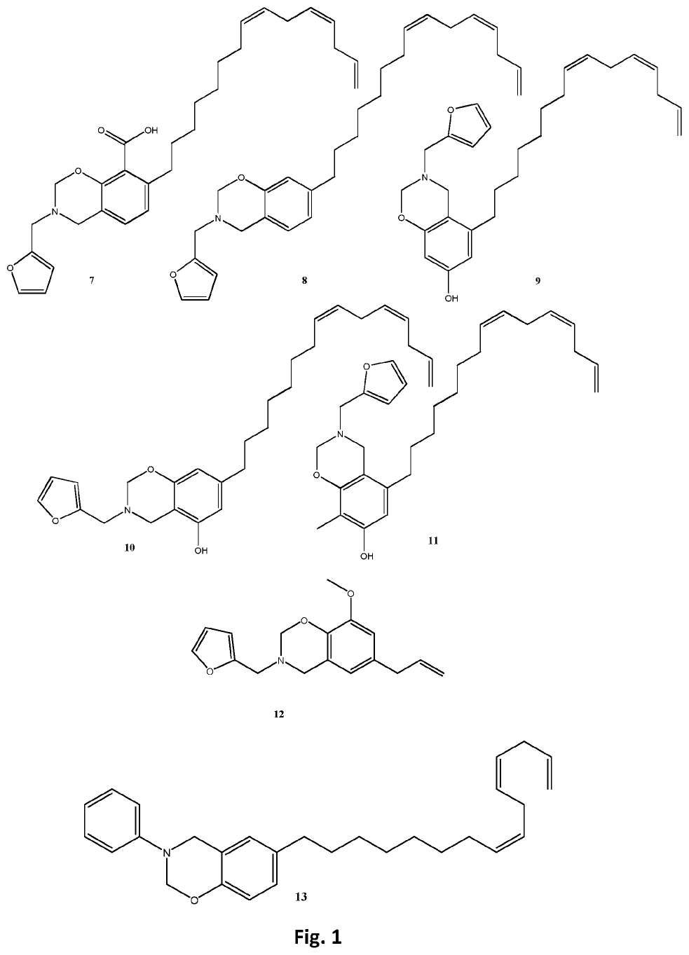

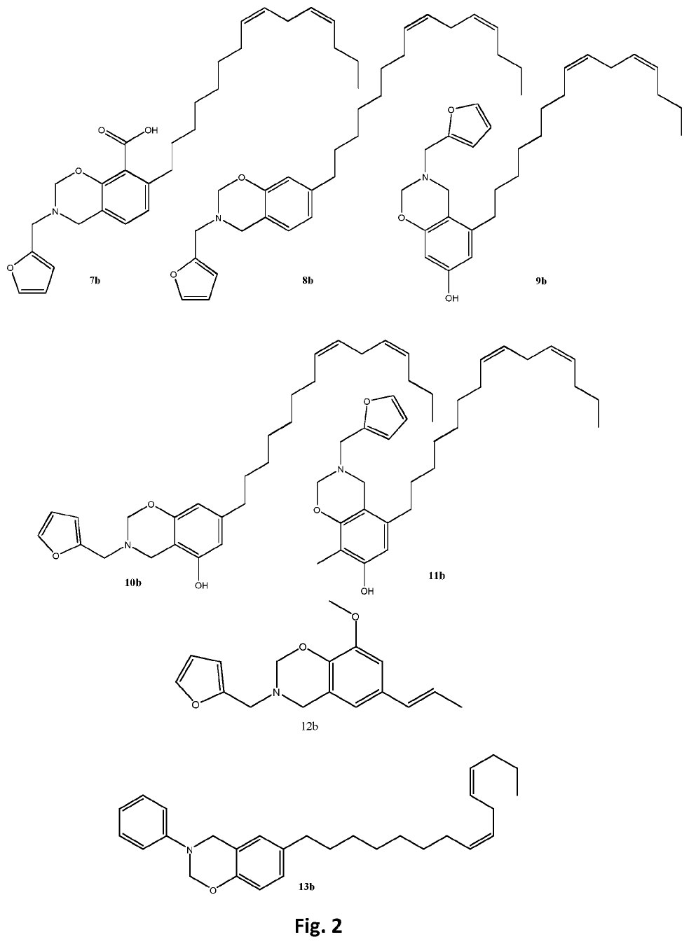

FIGS. 1-3 show the monomers 1-12 that are based on benzoxazines and that are used in the present invention. The monomer 13 is here used as a comparative example, not of the invention. By looking at the compounds 1-13 more carefully on FIG. 1, it is highlighted that the monomers 7, 8, 9, 10, 11, and 13 have a side chain that can vary in its degree of unsaturation. As shown on FIG. 2, the corresponding monomers 7b, 8b, 9b, 10b, 11b, and 13b have only two olefinic groups while the corresponding monomers 7c, 8c, 9c, 10c, 11c, and 13c have only one olefinic group as described on FIG. 3. Finally, the side chain can fully be saturated as depicted on FIG. 4 for the corresponding monomers 7d, 8d, 9d, 10d, 11d and 13d. It is precised that among those similar monomers, the monomer bearing three unsaturations is the major compound and the monomer bearing only one unsaturation is the minor compound. It is further noted that when one of this particular monomer is used, it is relatively difficult to know which one is used. For the purpose of the present invention, it is considered that when, for example compound 8 is used, compounds 8b, 8c and 8d are also comprised. It is finally precised that all the unsaturations shown in relation with those specific compounds have the configuration Z (or cis).

Starting from 8-tocopherol as a phenol derivative starting material, the following reactional scheme has been established (scheme I).

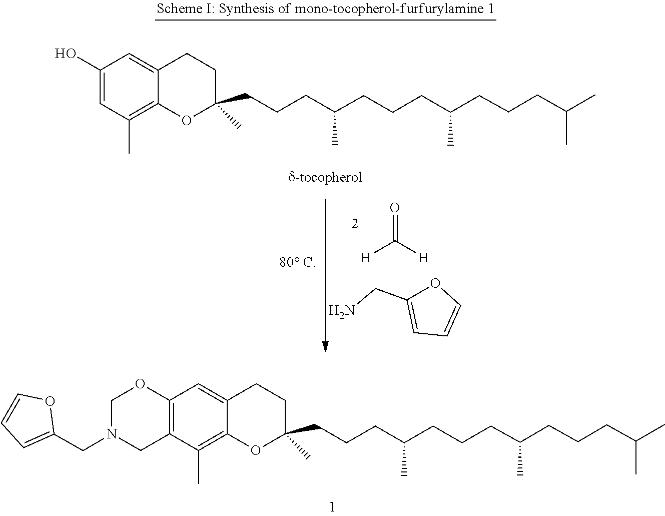

This solventless reaction is performed during 24 h at 80° C. The product is then purified by liquid-liquid extractions from CHCl₃ in 1M NaOH (in triplicate) and water (in triplicate). The mixture is then dried with MgSO₄, filtered, and dried under reduced pressure at 50° C. No further purification has been required. The overall yield of the reaction has been determined to be equal of 96%. The state of the monomer 1 at room temperature is a viscous liquid.

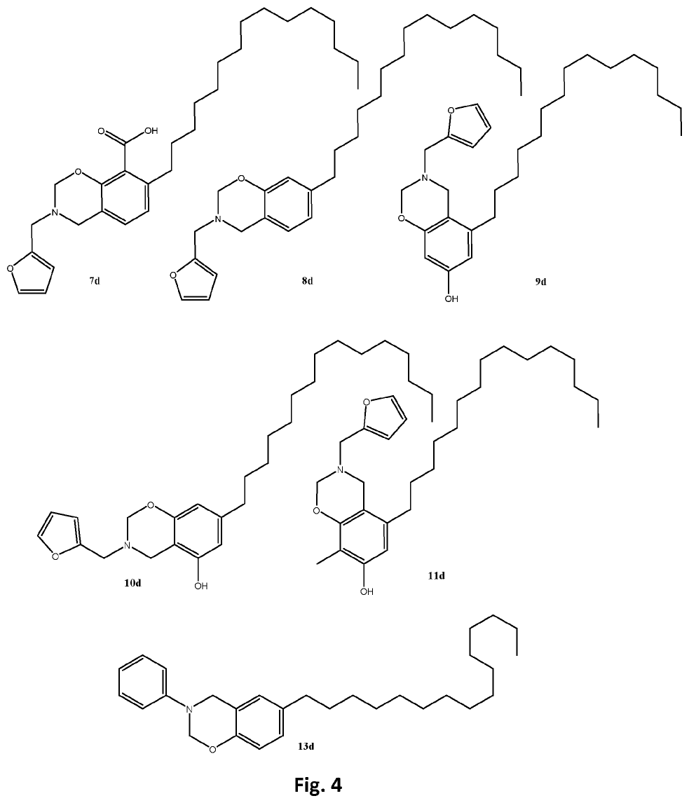

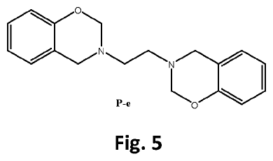

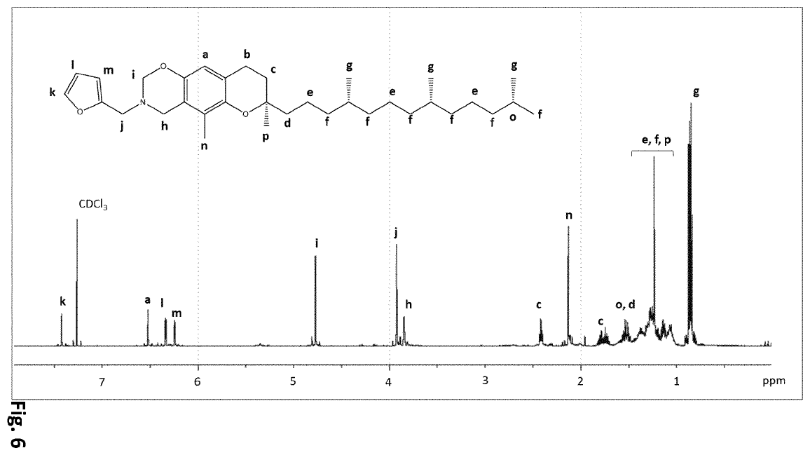

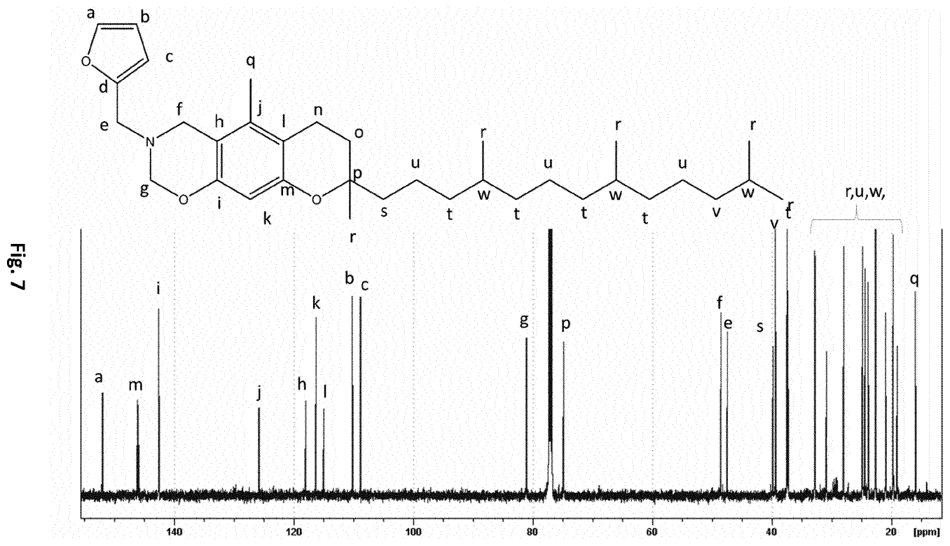

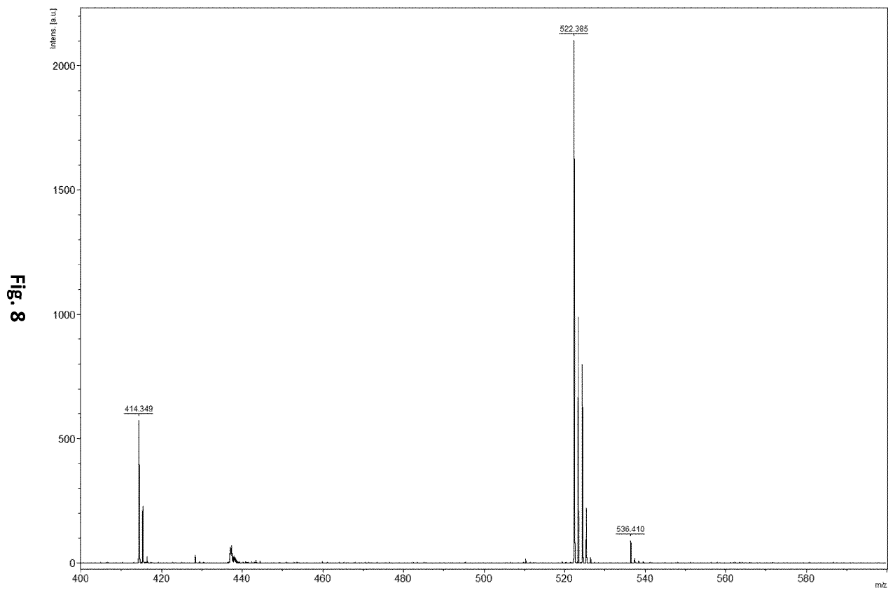

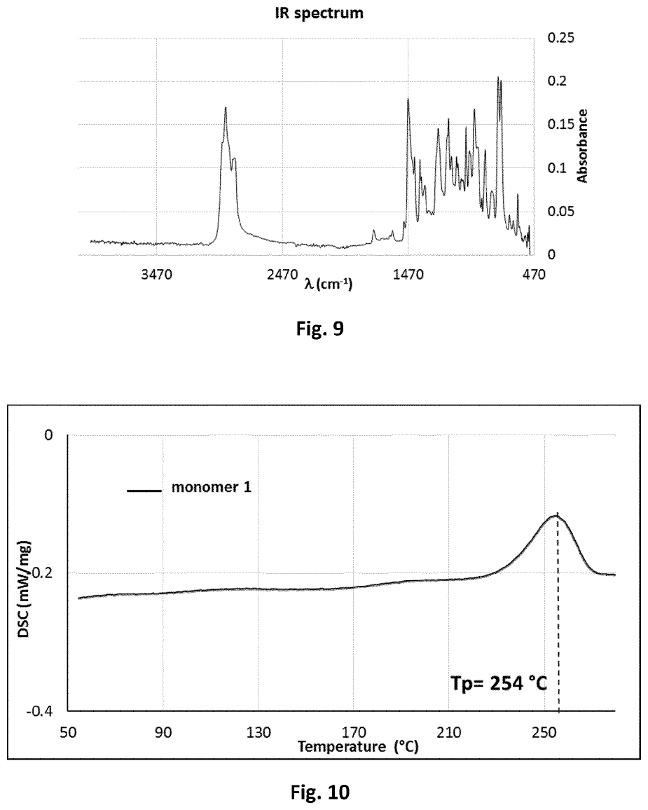

FIGS. 6-9 show the analytical data of the compound 1 (respectively ¹H and ¹³C NMR, mass and IR spectra).

### Study of the Polymerisation Behaviour

FIG. 10 shows the results of the DSC (Differential Scanning Calorimetry) analysis of monomer 1. The DSC analysis has been performed in the following temperature range: 25° C. to 280° C. The DSC traces were recorded at a heating rate of 5° C./min under nitrogen. The polymerization temperature (Tₚ) has thus been determined to be equal to 254° C. (ring opening of the oxazine moiety of the monomer 1).

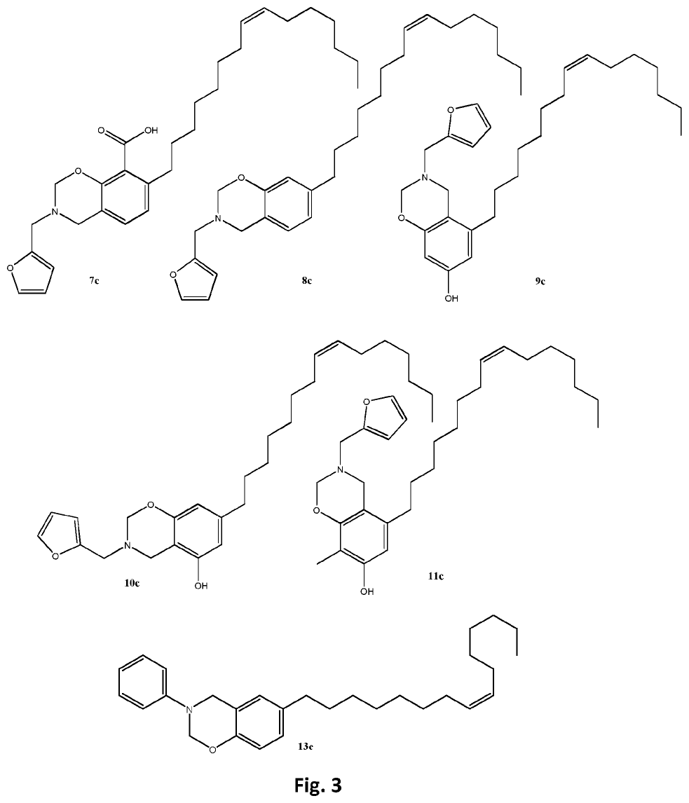

Since monomers 1-13 are mono-functional, their polymerization is not sufficient for the material to be efficiently cross-linked and to form an effective self-supported coating. In order to study the behaviour of these monomers for coating experiment, a DSC analysis on a composition comprising the monomer 1 and P-e (3,3′-ethane-1,2-diylbis(3,4-dihydro-2H-1,3-benzoxazine) has been performed. P-e is a bi-functional compound (see chemical structure on FIG. 5), presenting two oxazine rings.

P-e has been synthesized following a known protocol (Ganfoud R. et al., Composites Sci. Technol., 2015, 110, 1-7; Puchot L. et al, Polymer Chemistry, 2018, 9, 472).

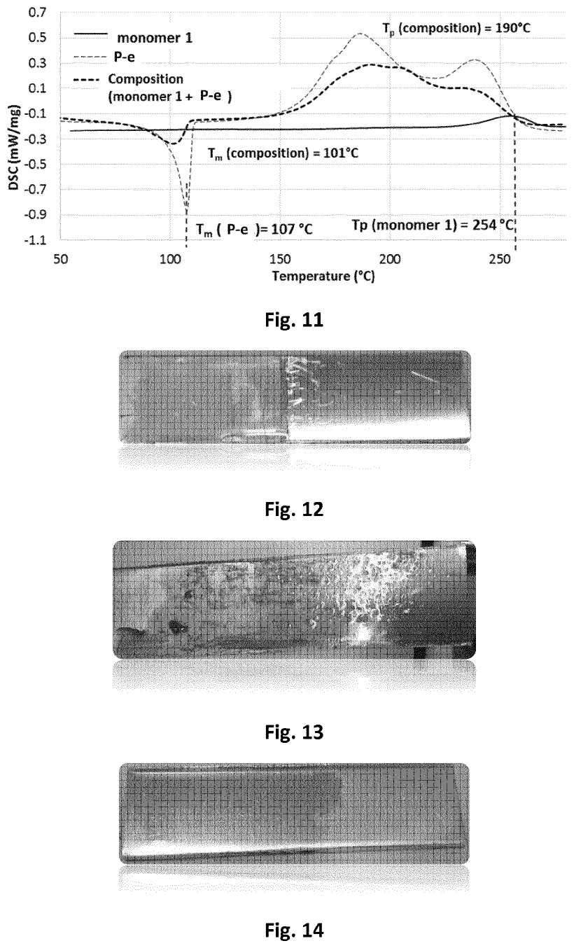

The composition comprises 33% of monomer 1 (17 mg) and 67% of P-e (34 mg) and the mixing is achieved at 100° C. A pasty solid with an orange color is obtained. FIG. 11 shows the DSC curves obtained on the composition. The range of temperature is 25° C. to 280° C. and the heating rate is of 5° C./min under nitrogen.

The DSC curve of the P-e alone shows a melting point at 107° C. and two exothermic peaks (186° C. and 238° C., not indicated as such on FIG. 11) identifying the successive ring-opening of the two benzoxazine rings. The aspect of the DSC curve for the composition shows that the polymerisation of P-e seems to occur in the first place and that the polymerisartion with the monomer 1 follows.

### Coating Experiments

### Coating of P-e

A coverslip in steel is treated with NaOH_{(aq)} in order to favour the adherence of the polymer film. A drop of P-e (obtained by melting the P-e at 107° C.) is deposited and spread out on the coverslip. The coverslip is then placed in the sterilizer at 180° C. for 2 h in order to favour the polymerisation and the formation of the coating. Finally, the discs were sterilised by soaking them in 70% ethanol in water for 30 min.

FIG. 12 shows a picture of the coating of P-e on a steel coverslip that has been treated with NaOH. The thickness is about 120 μm.

The coating is smooth and homogeneous.

### Coating of the Composition (Monomer 1+P-e)

A coverslip in steel is treated with NaOH_{(aq)} in order to favour the adherence of the polymer film. A drop of the composition (obtained by melting the composition at 110° C.) is deposited and spread out on the coverslip. The coverslip is then placed in the sterilizer at 180° C. for 2 h in order to favour the polymerisation and the formation of the coating. Finally, the substrates were sterilized by soaking them in 70% ethanol in water for 30 min.

FIG. 13 shows a picture of the coating of the composition on a steel coverslip that has been treated with NaOH.

The result is less satisfying than the result obtained with P-e alone, since the coating shown on the picture of FIG. 13 is inhomogeneous and rough, in comparison to the coating shown on the picture of FIG. 12.

Coating of the Composition (Monomer 1+P-e) without a Pre-Treatment of the Coverslip.

This time, the coating is performed on a coverslip in steel that has not been treated. A drop of the composition (obtained by melting the composition at 110° C.) is deposited and spread out on the coverslip. The coverslip is then placed in the sterilizer at 180° C. for 2 h in order to favour the polymerisation and the formation of the coating. Finally, the substrates were sterilized by soaking them in 70% ethanol in water for 30 min. The thickness of the coating is is about 110 μm.

FIG. 14 shows a picture of the coating of the composition on a steel coverslip that has not been treated with NaOH.

These coating experiments have demonstrated that the substrate can be used without any pre-treatment.

The hardness of the coating was measured using the Pencil Hardness Tester Elcometer 501, in accordance with the following standard ASTM D 3363. A scratch on the coating was not observed using pencil with a hardness lower than 5H.

In order to perform the polymerisation at a temperature inferior to 180° C., the use of Lewis acid as catalyst can be made. Such Lewis acid catalyst are in various instances PCl₅, PCl₃, POCl₃, TiCl₄, AlCl₃ or CH₃OTf. They can be added at concentration varying between 0.5 wt % and 10 wt %. These compounds are added within the composition that is deposited onto the surface of the substrate and are washed out during the soaking in ethanol. The temperature which is then employed can be lowered until 150° C. The range of temperature when these catalysts are present thus vary between 150° C. and 180° C.

To lower the polymerisation temperature, primary amines, quaternary ammoniums, thiols and elemental sulphur can be used. These compounds are added within the composition that is deposited onto the surface of the substrate and are washed out during the soaking in ethanol. The thickness of the film is about 120 μm.

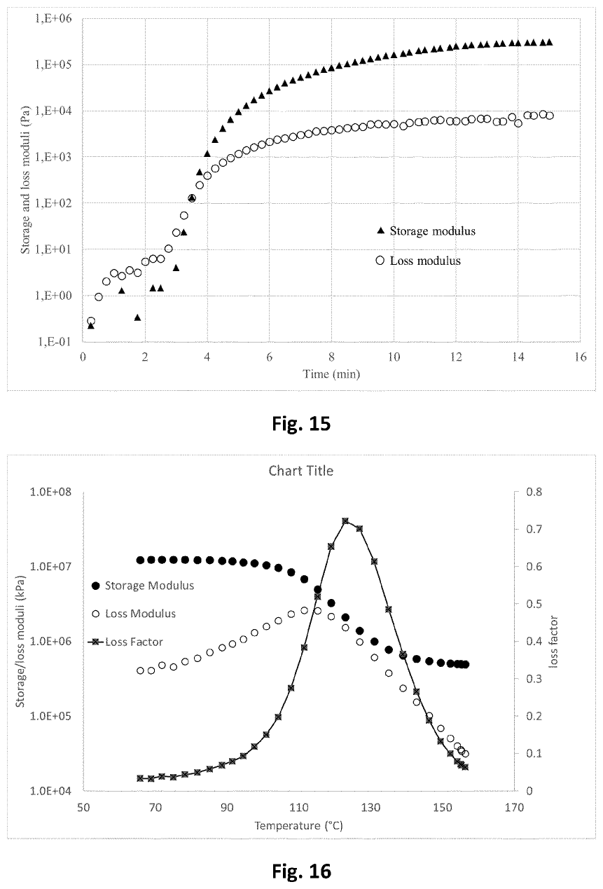

FIG. 15 shows a rheogram. Rheo-kinetic measurements were performed with an Anton Paar Physica MCR 302 rheometer equipped with a CDT 450 temperature control device with disposable aluminium plate-plate (diameter 25 mm, measure gap 0.35 mm) geometry. The polymerization measurements were recorded in oscillation mode at imposed 1% strain amplitude (γ) and a frequency (f) of 1 Hz. A heating ramp of 20° C./min was applied to reach the temperature of 150° C. Gelation points were measured at 150° C. whereas the sample deformation was ramped linearly from 1% to 0.2% to remain within the instrument limitation and to maintain linear viscoelastic behaviour as the moduli increase over several orders of magnitude upon curing. Gelation point corresponds to the time needed at this temperature to reach the crossing of the storage and loss moduli. The measurement show a solid material is formed after 200 seconds at 150° C.

FIG. 16 shows a rheogram of the final cross-linked polymer consisting of monomer 1 and P-e. Rheo-kinetic measurements were performed with an Anton Paar Physica MCR 302 rheometer equipped with a CDT 450 temperature control device with disposable aluminium plate-plate (diameter 25 mm, measure gap 0.35 mm) geometry on a sample pre-crosslinked between the plates of the rheometer, as described above. The measurements were recorded in oscillation mode at imposed 0.05% strain amplitude (γ) and a frequency (f) of 1 Hz. A heating ramp of 20° C./min was applied to reach the temperature of 150° C. The mechanical relaxation temperature corresponds to the maximum of the loss factor, at 122° C.

### Pharmaceutical Aspect

### Film Preparation

Tocopheryl-benzoxazine 1 and P-e were provided according to the above mentioned protocol. Poly(L-Lysine) (cat. #P2636) was purchased from Sigma-Aldrich (UK).

Monomers, in 1:2 weight ratio (monomer 1:P-e), were dissolved in chloroform at a total final concentration of 20% w/v. Alternatively, other aprotic solvents such as acetone or dichloromethane can be employed. Still alternatively, the monomers in 1:2 weight ratio (monomer 1:P-e) have been melted together at a temperature ranging between 105° C. and 110° C. This second way to mix both monomer 1 and P-e allows for preventing the use of chloroform. Approximately 100 mg of the solution or of the molten mixture were deposited on ø=13 mm circular glass coverslips placed on a stirring plate, which was then kept at 100° C. for 15 min. The thermal treatment was performed at 150° C. for 1 h followed by a post-cure at 170° C. for 2 h. Finally, the discs were sterilised by soaking them in 70% ethanol in water for 30 min. The thickness of the film is about 110 μm.

In addition to clear glass coverslips, as a control, also glass coverslips coated with poly-L-lysine were used. In particular, 50 μL of a sterile 0.01% w/v poly-L-lysine solution in MilliQ water were deposited on the coverslips and after 10 min the solution was removed and the glass surface was rinsed with MilliQ water. Coverslips were allowed to dry for at least 2 h before introducing cell culture medium.

### Cellular Studies

General cell culture. The mouse leukaemic monocyte macrophage cell line RAW 264.7 and the human monocyte cell line THP-1 (TIB-202™) were purchased from ATCC (Manassas, Va., USA) and maintained in cell culture media (Dulbecco's Modified Eagle Medium (DMEM) high glucose (#D5671) for RAW 264.7, RPMI 1640 Medium (#R0883 for THP-1) supplemented with 10% (v/v) foetal bovine serum (FBS, #F7524), 2 mM L-Glutamine (#G7513), and 1% (v/v) penicilin-streptomycin (#P4333). All cell culture reagents were purchased from Sigma-Aldrich (UK) unless specified otherwise. Cells were routinely cultured in a humidified 5% (v/v) CO₂ air atmosphere at 37° C. and used to maximum passage number of 15.

THP-1 differentiation (M0 macrophages). THP-1 premonocytes of passages below 20 (1.25×105 cells/cm²) were differentiated into THP-1 macrophages for 24 h by incubation in complete medium containing 50 ng-mL-1 phorbol 12-myristate 13-acetate (PMA; #P1585, Sigma-Aldrich, UK). (See de la Rosa J. M. R. et al., Adv. Healthcare Mater, 2017, 1601012 for the protocol). After differentiation, PMA medium was removed; cells were washed once with serum-free RPMI 1640 and rested or further activated for 24 hours in PMA-free complete medium.

Metabolic activity. RAW 264.7 macrophages were seeded on tissue culture polystyrene (TCPS), glass, or polymer films (poly-L-lysine or tocopherol) placed in the wells of 24-well plates at a density of 2.5×104 cells/cm² and allowed to grow for 48 h. THP-1 premonocytes were differentiated and rested on the substrates as described above in order to obtain THP-1 macrophages. Cells were then washed with PBS and incubated for one hour at 37° C. in serum- and phenol red-free medium containing CellTiter 96® AQueous One Solution Cell Proliferation Assay (MTS) 5% (v/v). Metabolic activity was measured by reading the absorbance values at 490 nm (Synergy2 Biotek plate reader, using Gen5 software) and normalized by the amount of total protein content in each well using the BCA kit (Sigma-Aldrich, UK).

Effect of substrate on the production of inflammatory mediators. RAW 264.7 macrophages were seeded on tissue culture polystyrene (TCPS), glass, or polymer films (polylysine or tocopherol) placed in the wells of 24-well plates at a density of 2.5×104 cells/cm² and allowed to grow for 24 h. RAW 264.7 macrophages were treated for 24 hours with fresh medium containing 1 μg/mL Lipopolysaccharides (LPS) from Escherichia coli 026:B6 (L5543, Sigma-Aldrich, UK) alone. Freshly differentiated THP-1 macrophages were treated for 24 h with fresh medium containing 100 ng/mL LPS plus 20 ng/mL IFN-γ (#300-02, Prepotech, Inc.). RAW 264.7 and THP-1 cells cultured in fresh medium without any effectors were used as a negative control. After activation, supernatants were collected and centrifuged at 13,000 rpm for 5 minutes. For RAW 264.7 macrophages the amount of TNF-α in culture medium was determined by BD OptEIA™ mouse TNF (Mono/Mono) ELISA set according to manufacturer instructions. For THP-1 macrophages the amount of TNF-α was determined by TNF-α ELISA MAXM Deluxe (#43020, BioLegend, UK) according to manufacturer instructions. TNF-α levels were normalized by the amount of total protein content in each well by using the BCA kit.

FIG. 17 shows the effect of the substrate on the metabolic activity in RAW 264.7 (left) and THP-1 (right) macrophages. The concentration values are normalized against the total amount of protein of the sample, which is assumed to be roughly proportional to the cells number.

It has been observed that there are not significant difference in metabolic activity between macrophages exposed to tissue culture polystyrene (TCPS), glass discs (uncoated or coated with poly-L-lysine) or polymer films (comprising notably the monomer 1) deposited on glass substrate. The viability of the macrophages is thus maintained.

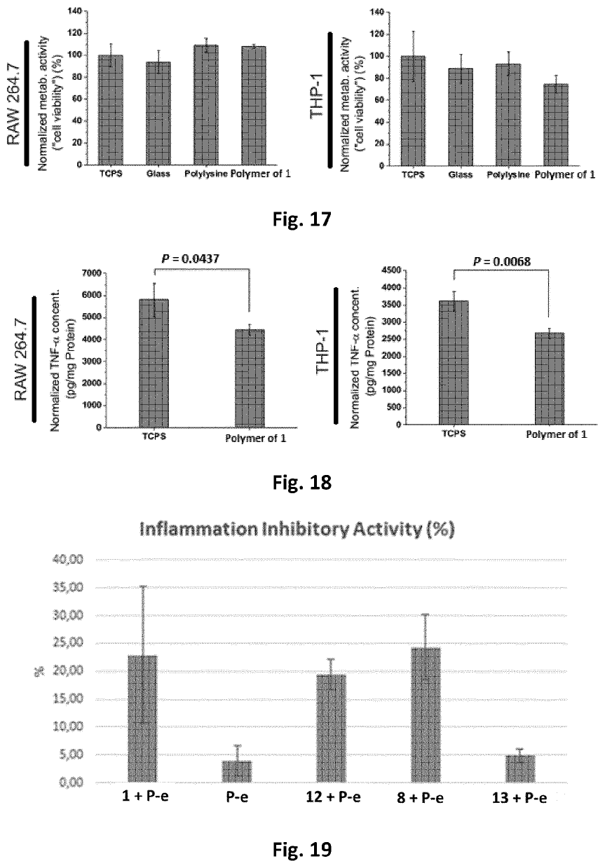

FIG. 18 shows the effect of the substrate on the TNF-α production in RAW 264.7 (left) and THP-1 (right) macrophages. The concentration values are normalized against the total amount of protein of the sample, which is assumed to be roughly proportional to the cell number.

The TNF-α production in macrophages seeded either on tissue culture polystyrene (TCPS) or polymer film (comprising notably the monomer 1) has been measured by ELISA after 24 h incubation with LPS (1 μg/mL) for RAW 264.7 and with LPS (100 ng/mL) plus IFN-γ (20 ng/mL) for THP-1.

### Anti-Inflammatory Effect

Compounds 1, 8, 12, and 13 (comparative example) in combination with P-e and P-e alone have been tested with respect to their anti-inflammatory properties.

In human, inflammation can arise through the activation of nuclear factor-kappaB (NF-κB), a pivotal transcription factor in chronic inflammatory diseases, inducing the expression of numerous inflammation related-genes such as those for tumor necrosis factor α (TNFα) and cytokines such as IL-1β, IL-10, and IL-6 or IL-8 (Medzhitov R., Nature, 2008, 454, 428-435). In the present invention, an inflammation-relevant human cell line, i.e. the monocytes THP-1 cells, are used to evaluate the potential of the coatings to reduce the inflammation. Their cytotoxicity towards are first evaluated using the resazurin reduction test (O'Brien J. et al., Eur. J. Biochem., 2000, 267, 5421-5426.) General anti-inflammatory properties are then determined using THP-1 cells transfected with a reporter plasmid expressing the secreted alkaline phosphatase (SEAP) gene under the control of an NF-κB-inducible promoter. TNFα are used as an inflammation inducer and the data are expressed as percentage inhibition of NF-κB activation. In addition, the effects of the most promising monomers is further evaluated on inflammation-related genes by real time qPCR using THP-1 cells differentiated into macrophages (Andre C. M. et al., J. Agric. Food. Chem, 2013, 61, 2773-2779) and IL-1β, IL-10, IL-6, and COX-2 as markers (Kaulmann A. et al., Mol. Nutr. Food Res., 2016, 60, 992-1005).

FIG. 19 shows that the samples coated with a polymer formed from the monomers 1, 8, and 12 (the thickness of the film is about 120 μm) have a strong anti-inflammatory effect, in comparison with the samples coated with a compound P-e or 13 (not of the invention). Roughly, the inhibition of the inflammation amounts to 20%. The high variability for the coating resulting from the composition comprising monomer 1 and P-e can be explained by a difference in the quantity of coating or by biological variability.

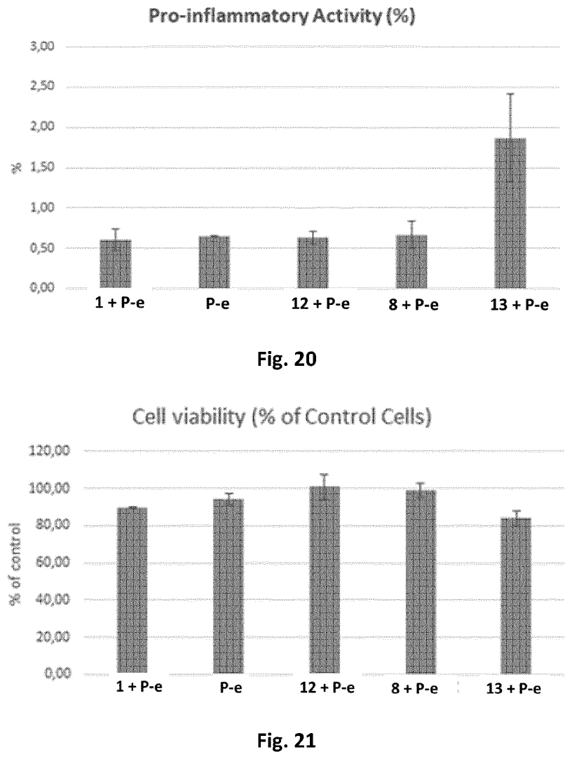

FIG. 20 shows the pro-inflammatory properties of the five compounds 1, P-e, 8, 12, and 13. In fact, only the sample coated with the film derived from compound 13 shows a trend to provoke the inflammatory response of the host.

Moreover, none of the five compounds 1, P-e, 8, 12, and 13 is toxic with regards to the biological cells, as shown in FIG. 21.

### Anti-Oxidant Effect

Once polymerized upon heating, polybenzoxazines exhibit a phenolic structure, which is highly suspected to exhibit a radical scavenging activity, and ensuing anti-oxidant properties.

A wide range of both in vivo and in vitro methods are currently used to assess the antioxidant activity of compounds or polymers, all of which having certain advantages and limitation. Here, both the oxygen radical absorbance capacity (ORAC) method and 2,2-Diphenyl-1-picrylhydrazyl (DPPH) Radical Scavenging Capacity Assay has been used to get a more complete picture of its antioxidant potential. In the ORAC assay, the peroxyl radical reaction involves hydrogen atom transfer mechanisms, whereas the DPPH assay is based on an electron transfer. Briefly, for ORAC analyses, 2,2′-azobis(2-amidinopropane) dihydrochloride (AAPH), i.e. a water-soluble azo compound, is used as a peroxyl radical generator; trolox, i.e. a water-soluble tocopherol analogue, is used as standard; and fluorescein is used as fluorescent probe. The fluorescence is measured every minute for 50 min. All samples are analyzed in triplicate and the final ORAC values are calculated using the net area under the decay curves as described (Andre C. M., et al., J. Agric. Food Chem., 2007, 55, 366-378). In the DPPH assay the capacity of the different building blocks to reduce an oxidant (an organic nitrogen radical which changes colour when reduced) is monitored by spectrophotometry at 515 nm.

Studies performed with polymer P-e alone (comparative example) and with polybenzoxazines formed especially from the monomers 1, 8, and 12, with or without P-e, show that anti-oxidant properties, without being limited, are enhanced by 5% to 25% when polybenzoxazines of the invention are used in comparison to P-e.

### Implementation of the Coatings of the Present Invention

These interesting properties (anti-inflammatory, anti-oxidant, low trend to be pro-inflammatory and absence of toxicity) are ideal for all the implementation relating to implantable metallic apparatus, the implantable metallic apparatus being in various instances a catheter, a metallic implant, or a metallic prosthesis.

### Experimental Procedure for the Synthesis of Monomers 1 to 12

Monomer 1

| | | | Para- | | | |
|---|---|---|---|---|---|---|
| | δ-Tocopherol | | formaldehyde | | Furfurylamine | |
| mol | 0.0018 | mol | 0.0036 | | 0.0018 | mol |
| molar | 402.65 | g · mol⁻¹ | 31.83 | g · mol⁻¹ | 97.12 | g · mol⁻¹ |
| mass | | | | | | |
| mass | 0.725 | g | 0.115 | g | 0.175 | g |

The reaction is done without solvent. Furfurylamine, tocopherol and paraformaldehyde are all weighted in a flask. The mixture is stirred and heated at 80° C. for 24 h without reflux. The product is purified by liquid-liquid extractions from CHCl₃ in 1M NaOH (in triplicate) and water (in triplicate). The mixture is then dried with MgSO₄, filtered, and dried under reduce pressured at 50° C. The yield of the reaction is 96%.

Monomer 2

| | Delta | | Para- | | | |
|---|---|---|---|---|---|---|
| | tocotrienol | | formaldehyde | | Furfurylamine | |
| mol | 0.0018 | mol | 0.0036 | mol | 0.0018 | mol |
| molar | 396.62 | g · mol⁻¹ | 31.83 | g · mol⁻¹ | 97.12 | g · mol⁻¹ |
| mass | | | | | | |
| mass | 0.714 | g | 0.115 | g | 0.175 | g |

The reaction is done without solvent. Furfurylamine, Delta tocotrienol and paraformaldehyde are all weighted in a flask. The mixture is stirred and heated at 80° C. for 24 h without reflux. The product is purified by liquid-liquid extractions from CHCl₃ in 1M NaOH (in triplicate) and water (in triplicate). The mixture is then dried with MgSO₄, filtered, and dried under reduce pressured at 50° C.

Monomer 3

| | beta | | Para- | | | |
|---|---|---|---|---|---|---|
| | tocophenol | | formaldehyde | | Furfurylamine | |
| mol | 0.0018 | mol | 0.0036 | mol | 0.0018 | mol |
| molar | 416.68 | g · mol⁻¹ | 31.83 | g · mol⁻¹ | 97.12 | g · mol⁻¹ |
| mass | | | | | | |
| mass | 0.750 | g | 0.115 | g | 0.175 | g |

The reaction is done without solvent. Furfurylamine, beta tocophenol and paraformaldehyde are all weighted in a flask. The mixture is stirred and heated at 80° C. for 24 h without reflux. The product is purified by liquid-liquid extractions from CHCl₃ in 1M NaOH (in triplicate) and water (in triplicate). The mixture is then dried with MgSO₄, filtered, and dried under reduce pressured at 50° C.

Monomer 4

| | beta | | Para- | | | |
|---|---|---|---|---|---|---|
| | tocotrienol | | formaldehyde | | Furfurylamine | |
| mol | 0.0018 | mol | 0.0036 | mol | 0.0018 | mol |
| molar | 410.63 | g · mol⁻¹ | 31.83 | g · mol⁻¹ | 97.12 | g · mol⁻¹ |
| mass | | | | | | |

| | | | | | |
|---|---|---|---|---|---|
| mass | 0.739 | g | 0.115 | g | 0.175 |

The reaction is done without solvent. Furfurylamine, beta tocotrienol and paraformaldehyde are all weighted in a flask. The mixture is stirred and heated at 80° C. for 24 h without reflux. The product is purified by liquid-liquid extractions from CHCl₃ in 1M NaOH (in triplicate) and water (in triplicate). The mixture is then dried with MgSO₄, filtered, and dried under reduce pressured at 50° C.

Monomer 5

| | gamma | | Para- | | | |
|---|---|---|---|---|---|---|
| | tocophenol | | formaldehyde | | Furfurylamine | |
| mol | 0.0018 | mol | 0.0036 | mol | 0.0018 | mol |
| molar | 416.68 | g · mol⁻¹ | 31.83 | g · mol⁻¹ | 97.12 | g · mol⁻¹ |
| mass | | | | | | |

| | | | | | |
|---|---|---|---|---|---|
| mass | 0.750 | g | 0.115 | g | 0.175 |

The reaction is done without solvent. Furfurylamine, gamma tocophenol and paraformaldehyde are all weighted in a flask. The mixture is stirred and heated at 80° C. for 24 h without reflux. The product is purified by liquid-liquid extractions from CHCl₃ in 1M NaOH (in triplicate) and water (in triplicate). The mixture is then dried with MgSO₄, filtered, and dried under reduce pressured at 50° C.

Monomer 6

| | gamma | | Para- | | | |
|---|---|---|---|---|---|---|
| | tocotrienol | | formaldehyde | | Furfurylamine | |
| mol | 0.0018 | mol | 0.0036 | mol | 0.0018 | mol |
| molar | 410.63 | g · mol⁻¹ | 31.83 | g · mol⁻¹ | 97.12 | g · mol⁻¹ |
| mass | | | | | | |

| | | | | | |
|---|---|---|---|---|---|
| mass | 0.739 | g | 0.115 | g | 0.175 |

The reaction is done without solvent. Furfurylamine, gamma tocophenol and paraformaldehyde are all weighted in a flask. The mixture is stirred and heated at 80° C. for 24 h without reflux. The product is purified by liquid-liquid extractions from CHCl₃ in 1M NaOH (in triplicate) and water (in triplicate). The mixture is then dried with MgSO₄, filtered, and dried under reduce pressured at 50° C.

Monomer 7

| | Anacardic | | Para- | | | |
|---|---|---|---|---|---|---|
| | acid | | formaldehyde | | Furfurylamine | |
| mol | 0.0018 | mol | 0.0036 | mol | 0.0018 | mol |
| molar | 342.47 | g · mol⁻¹ | 31.83 | g · mol⁻¹ | 97.12 | g · mol⁻¹ |
| mass | | | | | | |

| | | | | | |
|---|---|---|---|---|---|
| mass | 0.616 | g | 0.115 | g | 0.175 |

The reaction is done without solvent. Furfurylamine, Anacardic acid and paraformaldehyde are all weighted in a flask. The mixture is stirred and heated at 80° C. for 24 h without reflux. The product is purified by liquid-liquid extractions from CHCl₃ in 1M NaOH (in triplicate) and water (in triplicate). The mixture is then dried with MgSO₄, filtered, and dried under reduce pressured at 50° C.

Monomer 8

| | | | Para- | | | |
|---|---|---|---|---|---|---|
| | cardanol | | formaldehyde | | Furfurylamine | |
| mol | 0.0018 | mol | 0.0036 | mol | 0.0018 | mol |
| molar | 297.3 | g · mol⁻¹ | 31.83 | g · mol⁻¹ | 97.12 | g · mol⁻¹ |
| mass | | | | | | |
| mass | 0.535 | g | 0.115 | g | 0.175 | g |

The reaction is done without solvent. Furfurylamine, cardanol and paraformaldehyde are all weighted in a flask. The mixture is stirred and heated at 80° C. for 24 h without reflux. The product is purified by liquid-liquid extractions from CHCl₃ in 1M NaOH (in triplicate) and water (in triplicate). The mixture is then dried with MgSO₄, filtered, and dried under reduce pressured at 50° C. The yield of the reaction is 96%.

Monomers 9 and 10

| | | | Para- | | | |
|---|---|---|---|---|---|---|
| | Cardol | | formaldehyde | | Furfurylamine | |
| mol | 0.0018 | mol | 0.0036 | mol | 0.0018 | mol |
| molar | 315.4 | g · mol⁻¹ | 31.83 | g · mol⁻¹ | 97.12 | g · mol⁻¹ |
| mass | | | | | | |
| mass | 0.567 | g | 0.115 | g | 0.175 | g |

The reaction is done without solvent. Furfurylamine, cardol and paraformaldehyde are all weighted in a flask. The mixture is stirred and heated at 80° C. for 2 h without reflux. The product is purified by liquid-liquid extractions from CHCl₃ in 1M NaOH (in triplicate) and water (in triplicate). The mixture is then dried with MgSO₄, filtered, and dried under reduce pressured at 50° C.

Monomer 11

| | Methyl | | Para- | | | |
|---|---|---|---|---|---|---|
| | Cardol | | formaldehyde | | Furfurylamine | |
| mol | 0.0018 | mol | 0.0036 | mol | 0.0018 | mol |
| molar | 329.4 | g · mol⁻¹ | 31.83 | g · mol⁻¹ | 97.12 | g · mol⁻¹ |
| mass | | | | | | |
| mass | 0.592 | g | 0.115 | g | 0.175 | g |

The reaction is done without solvent. Furfurylamine, methyl cardol and paraformaldehyde are all weighted in a flask. The mixture is stirred and heated at 80° C. for 24 h without reflux. The product is purified by liquid-liquid extractions from CHCl₃ in 1M NaOH (in triplicate) and water (in triplicate). The mixture is then dried with MgSO₄, filtered, and dried under reduce pressured at 50° C.

Monomer 12

| | | | Para- | | | |
|---|---|---|---|---|---|---|
| | Iso-eugenol | | formaldehyde | | Furfurylamine | |
| mol | 0.0018 | mol | 0.0036 | mol | 0.0018 | mol |
| molar | 164.20 | g · mol⁻¹ | 31.83 | g · mol⁻¹ | 97.12 | g · mol⁻¹ |
| mass | | | | | | |
| mass | 0.30 | g | 0.115 | g | 0.175 | g |

The reaction is done without solvent. Furfurylamine, iso-eugenol and paraformaldehyde are all weighted in a flask. The mixture is stirred and heated at 80° C. for 24 h without reflux. The product is purified by liquid-liquid extractions from CHCl₃ in 1M NaOH (in triplicate) and water (in triplicate). The mixture is then dried with MgSO₄, filtered, and dried under reduce pressured at 50° C. The yield of the reaction is 92%.

1.-20. (canceled)

21. A monomer based on a 3,4-dihydro-2H-1,3-benzoxazine derivative having the formula 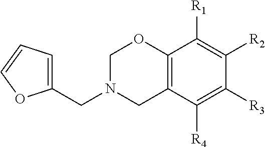
either wherein
R₄=CH₃
R₂ and R₃ together represent 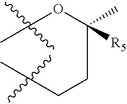
with R₅= 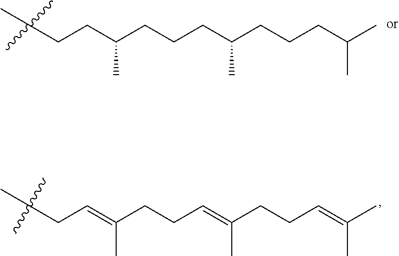 [SMILES: *CC[C@@H](C)CCC[C@@H](C)CCCC(C)C ; *C/C=C(\C)CC/C=C(\C)CCC=C(C)C]
and
R₁=H, or CH₃,
or wherein
R₄ and R₃ together represent 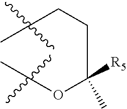
with 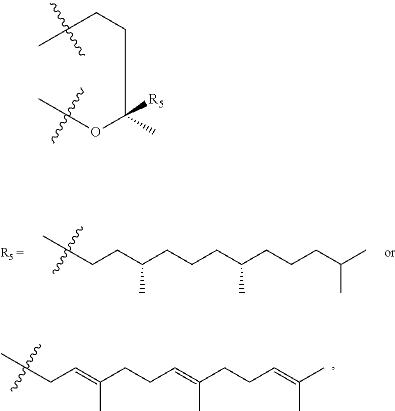
and
R₂=R₁=CH₃,
or a mixture thereof.

22. A composition comprising a mixture of a first monomer consisting of the 3,4-dihydro-2H-1,3-benzoxazine derivative monomer of any one of the formulas of claim 1, or a mixture thereof, and a second benzoxazine derivative selected from the group consisting of monofunctional amines bridged with diphenolic compounds, monophenolic compounds bridged with diamines and diamines bridged by diphenolic compounds, or a mixture thereof.

23. The composition according to claim 22, wherein the second benzoxazine derivative is a compound of formula A 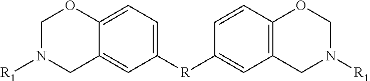
wherein R is a —CH₂—, —C(CH₃)₂—, SO₂, —C(CF₃)₂—, —C(CH₃)(C₆H₅)—, —C(CH₃)(C₂H₅)—, —C(C₆H₅)₂—, —CHCH₃—, —C₆H₁₀— or —C(CH₃)CH₂CH₂COOH— group;
R₁ is a —CH₂CH₂OH, vinyl, methyl, ethyl, propyl, isopropyl, butyl, hexyl, cyclohexyl, fluorene, phenylacetylene, phenyl propargyl ether, benzonitrile, furfuryl, phenylene or —(CH₂)₁₇CH₃ group.

24. The composition according to claim 22, wherein the second benzoxazine derivative is a compound of formula B 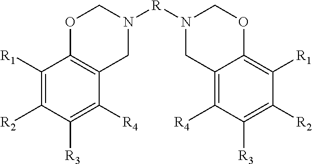
either wherein
R is a —(CH)₂—, —(CH)₄—, —(CH)₆—, —(CH)₈—, —(CH)₁₀—, —(CH)₁₂—, —(CH)₁₄—, or —(CH)₁₈— group,
or 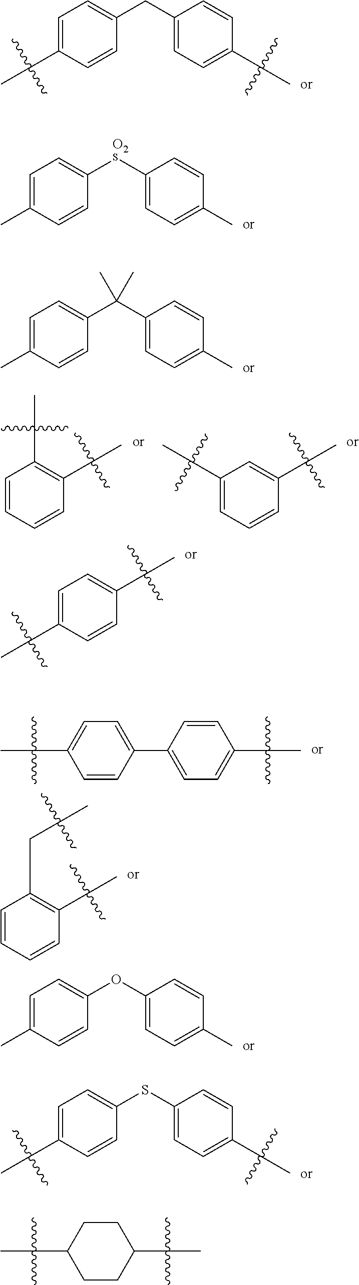
and wherein,
R₁, R₂, R₃, R₄ are selected from the group consisting of
R₁=R₂=R₃=R₄=H,
R₁=OCH₃, R₂=R₄=H, R₃=CH₂CH═CH₂,
R₁=OCH₃, R₂=R₄=H, R₃=CH═CHCH₃,
R₁=OCH₃, R₂=R₄=H, R₃=CHO,
R₁=OCH₃, R₂=R₃=R₄=H,
R₁=OCH₃, R₂=R₄=H, R₃=CHO,
R₁=OCH₃, R₂=R₄=H, R₃=CH₂CH₂COOH,
R₁=R₂=R₄=H, R₃=CH₂CH₂COOH,
R₁=R₂=R₄=H, R₃=CH═CHCOOH, and
R₁=OCH₃, R₂=R₄=H, R₃=CH═CHCOOH,
or wherein
R₃=R₄=H, 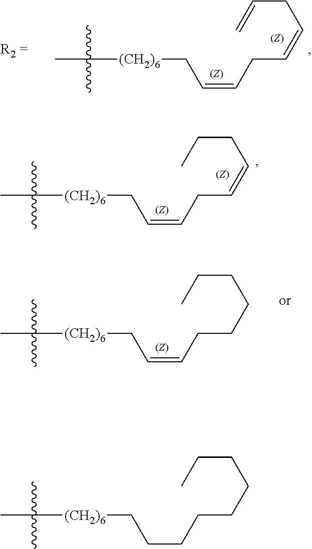
and R₁=COOH,
or wherein
R₁=H, R₂=OH, R₃=H
and 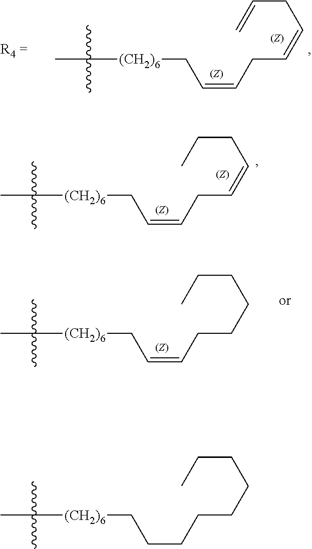
or wherein
R₁=H,
and 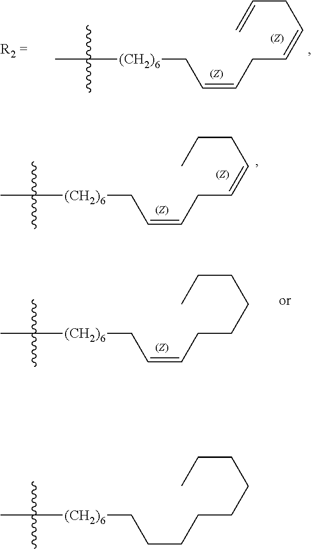
and R₃=H, R₄=OH,
or wherein
R₁=CH₃, R₂=OH, R₃=H 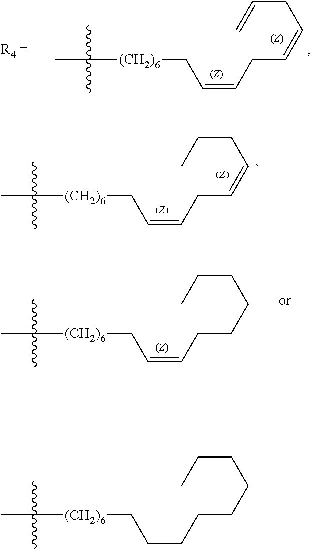
or wherein
R₁=R₃=R₄=H, and 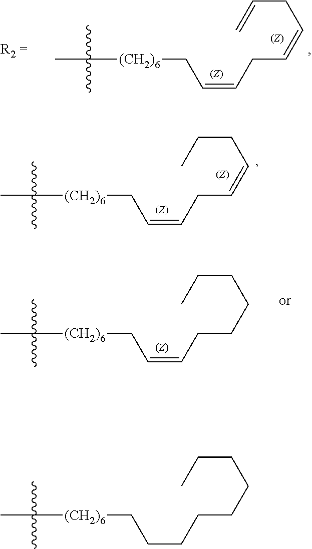
or wherein
R₁=R₃=H
R₂=—CH₂HC═CH₂, and R₄=OCH₃;
or
R₁=R₃=H
R₂=—CH═HCCH₃ and
R₄=OCH₃;
or R₁=R₂=R₄=H and 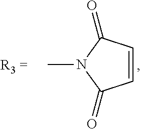
or R₁=R₂=R₃=H and R₄=CH₂CH═CH₂,
or R₁=R₂=R₄=H and R₃=CH₂CH═CH₂,
or R₁=R₃=R₄=H and R₂=CH₂CH═CH₂.

25. The composition according to claim 22, wherein in the composition the second benzoxazine derivative has a concentration equal or superior to 50% in comparison to the concentration of the first monomer.

26. A process for producing a material comprising a substrate coated with at least one of an anti-inflammatory and an anti-oxidant heterogeneous polymer based on a 3,4-dihydro-2H-1,3-benzoxazine derivative, the process comprising the steps of:
(a) depositing one of an anti-inflammatory and an anti-oxidant monomer selected from the group consisting of at least one monomer of the formulas of claim 1 and of further the formula 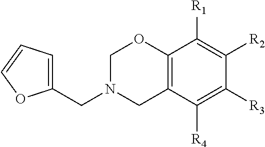
either wherein
R₃=R₄=H, 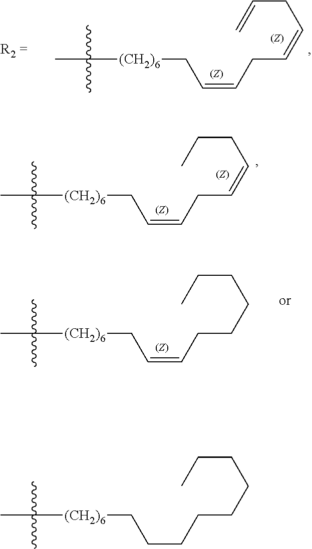
and R₁=COOH,
or wherein
R₁=H, R₂=OH, R₃=H
and 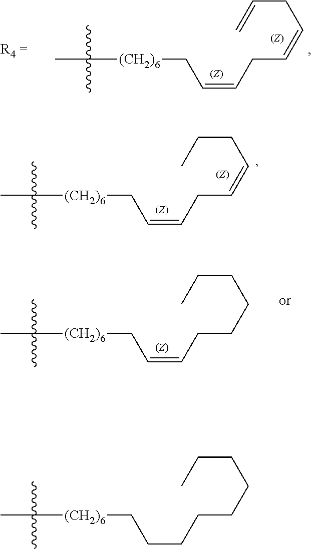
or wherein
R₁=H,
and 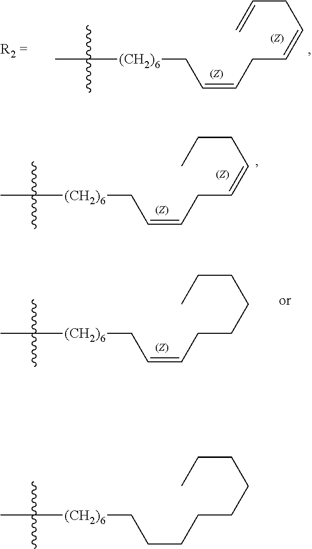
and R₃=H, R₄=OH,
or wherein
R₁=CH₃, R₂=OH, R₃=H 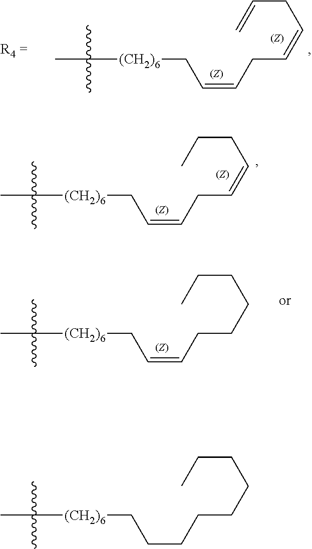
or wherein
R₁=R₃=R₄=H, and 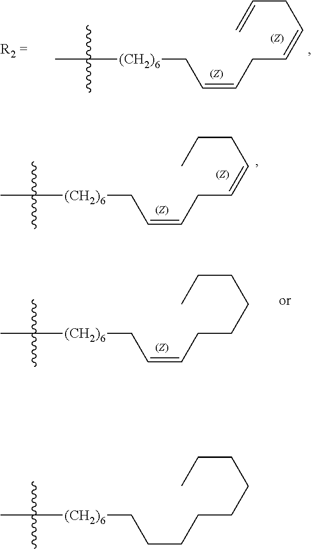
or wherein
R₂=R₄=H
R₃=—CH₂HC═CH₂ and
R₁=OCH₃;
or
R₂=R₄=H
R₃=—CH═HCCH₃ and
R₁=OCH₃,
or a mixture thereof onto the substrate,
(b) heating a deposit obtained through step (a) at a predetermined temperature and for a predetermined duration to polymerize the monomer to obtain the material.

27. The process according to claim 26, wherein the at least one of the anti-inflammatory and the anti-oxidant monomer based on a 3,4-dihydro-2H-1,3-benzoxazine derivative, being a first monomer, the process comprising a step of depositing a second benzoxazine derivative selected from the group consisting of monofunctional amines bridged with diphenolic compounds, monophenolic compounds bridged with diamines and diamines bridged by diphenolic compounds, or a mixture thereof, onto the substrate deposited with the first monomer during step (a) for generating a mixture, before the step (b) of heating.

28. The process according to claim 26, including a step of adding a second benzoxazine derivative, selected from the group consisting of monofunctional amines bridged with diphenolic compounds, monophenolic compounds bridged with diamines and diamines bridged by diphenolic compounds, or a mixture thereof, to the at least one of the anti-inflammatory and the anti-oxidant monomer based on a 3,4-dihydro-2H-1,3-benzoxazine derivative, being a first monomer, and a step of mixing to form a resulting composition of the first monomer and second benzoxazine derivative, the resulting composition being deposited onto the substrate in the step (a).

29. The process according to claim 27, wherein the second benzoxazine derivative is a compound of formula A, as defined in claim 23.

30. The process according to claim 28, wherein the second benzoxazine derivative in the resulting composition has a concentration equal or superior to 50% in weight in comparison to the concentration of the first monomer.

31. The process according to claim 26, wherein step (b) of heating is performed at a temperature within a range of from 100° C. to 250° C.

32. A material comprising a substrate with at least one of an anti-inflammatory and an antioxidant heterogeneous polymer based on 3,4-dihydro-2H-1,3-benzoxazine coating or film, obtainable by the process claim 26.

33. The material according to claim 32, wherein the heterogeneous polymer presents a mechanical relaxation temperature corresponding to the maximum of the loss factor of from 100° C. to 300° C.

34. The material according to claim 32, wherein the coating or the film presents a thickness of from of from 100 μm to 2 mm.

35. The material according to claim 32, wherein the coating or the film presents a hardness of more than 5H measured according the ASTM D 3363 standard.

36. The material according to claim 32, as a part of implantable materials.

37. The material according to claim 32 for use for the treatment or the prevention of at least one of inflammatory and oxidant disease(s).

38. The material according to claim 32, wherein the coating or the film presents a thickness of from 100 μm to 500 μm.

39. The material according to claim 36, wherein the part of implantable materials are implantable metallic apparatus such as a catheter, a metallic implant, or a metallic prosthesis, biologically compatible.

40. The process according to claim 27, wherein the second benzoxazine derivative is a compound of formula B as defined in claim 24.
